(19) United States
(12) Patent Application Publication
(10) Pub. No.: US 2023/0332081 A1
(43) Pub. Date: Oct. 19, 2023

(54) DEVICE FOR BIOLOGICAL CULTURES

(71) Applicants: POLITECNICO DI MILANO, Milano (IT); OSPEDALE SAN RAFFAELE S.R.L., Milano (IT)

(72) Inventors: Gianfranco Beniamino FIORE, Milano (IT); Monica SONCINI, Milano (IT); Marco PIOLA, Milano (IT); Lorenzo Pietro COPPADORO, Milano (IT); Chiara FOGLIENI, Milano (IT); Maria LOMBARDI, Milano (IT)

(73) Assignees: POLITECNICO DI MILANO, Milano (IT); OSPEDALE SAN RAFFAELE S.R.L., Milano (IT)

(21) Appl. No.: 18/042,376

(22) PCT Filed: Aug. 13, 2021
(86) PCT No.: PCT/IB2021/057468

§ 371 Date: Feb. 21, 2023

(30) Foreign Application Priority Data
Aug. 25, 2020 (IT) 102020000020410

(51) Int. Cl. C12M 3/00

(52) U.S. Cl. CPC C12M 23/42; C12M 23/48

(57) ABSTRACT

A cartridge is adapted to house at least one biological sample therein, where the cartridge contains at least two overlapping layers, and the layers include at least one layer of highly hydrophobic, inert, and biocompatible material, with contact angle Θ_{c}≥90°, hydrophobic layer, and optionally, at least one layer of double-sided adhesive material, where in the absence of the at least one layer of double-sided adhesive material, the overlapping layers are connected together by chemical and/or physical bonding, where each of the overlapping layers has at least one inner hole that is pervious when the layers overlap one another, and the at least one inner hole is closed by the at least one biological sample, where loaded in the cartridge.

### BACKGROUND ART

The study of cellular and tissue behavior is important in many fields such as biology, medicine, pharmacology.

Cellular and tissue functions comprise a series of relationships and it is important to have systems which allow a study thereof in a context as close as possible to the physiological and/or pathological one, which are also inexpensive, modular, and allow using small volumes of reagents.

Microscopic analyses of cells are conventionally performed on single or multi-well microscope slides, or in single or multi-well Petri dishes. A strong limitation of the systems of the prior art is linked to poor modularity, such as the fact that the cells, once treated, for example with additives, or mechanical treatments, or other, can be subjected to only one type of analysis, such as that under the microscope, but cannot be further used. For example, it is not possible to perform marker expression and subsequent migration studies on the same sample.

It is the object of the present invention to provide a fluidic device for static and/or dynamic biological cultures which has a greater modularity than those of the prior art. It is a further object to provide a fluidic device adapted to be used in microscopy analysis on two sides of the same sample, analyses with immuno-biochemistry and molecular biology methods, where said analyses can be performed by recovering the sample, even following studies over time and/or exposure to different treatments, so that it is available for further studies and/or analyses.

### DESCRIPTION OF THE DRAWINGS

FIG. 1: (A, B, C) An embodiment of the cartridge: (A) perspective view of the 2 layers embedded in the cartridge; (B) vertical sectional view of the 2 layers embedded in the cartridge, loaded with a biological sample; (C) perspective view of the assembled cartridge. (D, E, F) A second embodiment of the cartridge: (D) perspective view of the 3 layers embedded in the cartridge; (E) vertical sectional view of the 3 layers loaded with a biological sample; (F) perspective view of the assembled cartridge. (G, H, I) A third embodiment of the cartridge: (G) perspective view of the 4 layers embedded in the cartridge; (H) vertical sectional view of the 4 layers embedded in the cartridge, loaded with a biological sample; (I) perspective view of the assembled cartridge; (J, K, L) Cartridge in the third embodiment: (J) vertical section and plan view of the 4 layers embedded in the cartridge, loaded with a biological sample supported on a membrane; (K) vertical section and plan view of the 4 layers embedded in the cartridge, loaded with two biological samples supported on a first and a second membrane; (L) vertical section and plan view of the 4 layers embedded in the cartridge, loaded with a self-supporting biological sample; (M) perspective and profile view with color key for hydrophobic layer, double-sided adhesive layer, membrane, self-supporting biological sample.

FIG. 2: An embodiment of the fluidic device according to the present invention, with a cartridge inserted into a support. (A) exploded, vertical sectional view, with positioning of the cartridge; (B) exploded perspective view; (C) exploded perspective view with cartridge inserted.

FIG. 3: An embodiment of the fluidic device according to the present invention, with a cartridge inserted into a support and well. (A) exploded, vertical sectional view, with positioning of the cartridge; (B) exploded perspective view; (C) exploded perspective view with cartridge inserted.

FIG. 4: Fluidic device after breaking along the weakening notches. (A) top view; (B) exploded perspective view and cartridge extraction.

FIG. 5: An embodiment of the fluidic device according to the present invention, with a cartridge inserted into a support. (A) exploded, vertical sectional view with positioning of the cartridge; (B) exploded perspective view, with cartridge insertion. Further embodiment of the fluidic device according to the present invention, where the channels are made both in the double-sided adhesive layer and in the rigid element: (C) exploded perspective view, with cartridge insertion. Further embodiment of the fluidic device according to the present invention, where the channels are made in the rigid element: (D) exploded perspective view, with cartridge insertion.

FIG. 6: An embodiment of the fluidic device according to the present invention, with cartridge inserted into a support. (A) partially exploded perspective view with cartridge inserted; (B) compact perspective view; (C) top view after breaking along the weakening notches. (D) exploded perspective view, after breaking along the weakening notches, with extraction of the cartridge.

FIG. 7: (A) Luer connectors on reversibly removable support; (B) Luer connectors on reversibly removable support and connected to the fluidic device in the embodiment shown in FIG. 4. (C, D) an embodiment of the fluidic device with connectors made on the profile of the device, perspective view.

FIG. 8: Diagrammatic vertical sectional view of an embodiment of a fluidic device for static culture when in use.

FIG. 9: Diagrammatic vertical sectional view of an embodiment of a fluidic device for dynamic culture, when in use, positioned on a microscope.

FIG. 10: Diagrammatic vertical sectional view of an embodiment of a fluidic device for dynamic culture, open after the controlled breakage.

FIG. 11: (A) images representative of good cell viability status during culture in the fluidic device according to the present invention, identified by vital staining of nuclei with the intercalating agent Acridine Orange (AO): Caco-2 intestinal epithelial cells (left), EA.hy926 endothelial cells (center) and primary human smooth muscle cells from internal mammary artery (IMASMC, right); (B) images representative of cell polarization/differentiation and phenotypic preservation. Cross-sections (x, y; x, z; y, z, 40× lens, ×2 zoom) acquired by confocal microscope of Caco-2 labeled for human epithelial antigen (HEA) are shown on the left. In the center, a representative immunofluorescence image at 20× magnification of a field of EA.hy926 labeled for CD31 is shown, on the right a field of IMASMC labeled for smooth muscle actin, alpha isoform (vascular isoform). In the images in B, the nuclei have been stained with DAPI; (C) three-dimensional renderings obtained from Z-series images in confocal microscopy representative of the different spatial organization of post-culture EA.hy926 endothelial cells for 3 months in the fluidic device according to the present invention, in the absence of membrane pretreatment, or following membrane pretreatment with fibronectin, gelatin, or gelatin-fibronectin, respectively; (D) histograms of gene expression (TP53) encoding the transcription factor p53 for Caco-2 (left) and for EA.hy926 (right). Each sample was run in triplicate in qRT-PCR and the values are shown as ΔCT versus Beta-actin reference gene.

FIG. 12: An embodiment of the fluidic device-support station complex. (A) Exploded perspective view. In the insert (B), exploded, vertical sectional view.

FIG. 13: An embodiment of the fluidic device-support station complex. (A) top view; insert (B) exploded, in vertical section; (C) exploded perspective view.

### DEFINITIONS

For the purpose of the present invention, highly hydrophobic material means a material the contact angle Θ_{c} of which is considerably greater than 90°, preferably greater than 100°, even more preferably greater than 105°. Highly hydrophobic materials are for example polyisobutylene (PIB, Θc=112.1°), polytetrafluoroethylene (PTFE, Teflon, Θc=109.2°), or polydimethylsiloxane (PDMS, Θc=107.2°). In addition to being highly hydrophobic, the exemplified materials are also inert and biocompatible.

Alternatively, highly hydrophobic material means any material made highly hydrophobic through a surface treatment adapted to achieve such a purpose. Double-sided adhesive material herein means a film coated, on both faces, with at least one adhesive substance, or a layer entirely made of at least one adhesive substance, or again a film the faces of which have the feature of being adhesive by selecting or adjusting the surface affinity of a material towards the material itself or another material.

In the embodiments not comprising said at least one layer of double-sided adhesive material, the necessary connection function between the layers is obtained by chemical and/or physical bonding.

In the present description, magnetic components are components made of materials selected from Iron, Cobalt, Nickel, metal alloys such as Mn—Bi, Nd—Fe—B, compounds such as NiFe₂O₃, Fe₃O₄, possibly covered with protective layers.

Static and/or dynamic biological cultures herein mean, merely by way of example, co-cultures of two or more cell types, monocultures, and 3D co-cultures on membrane or integrated in a three-dimensional matrix, tissue cultures, engineered tissue cultures.

Biological sample herein means material of human, animal, or plant biological origin. By way of example, commercially available lineage cells, primary cells isolated from living tissues, cellular constructs and/or engineered functional tissue constructs, as well as organ and tissue fragments taken from animal or plant models, or resulting from surgical biopsies of patients, form a biological sample.

Membrane herein means a membrane adapted to support a biological sample. By way of example, said membrane is a microporous, or nanoporous, or non-porous but permeable, or selectively permeable, or completely non-permeable, or deformable membrane.

Coverslip herein means a small, very thin plate made of a material such as to ensure optical access, optionally gas exchange. In a preferred embodiment, it is a classic glass coverslip.

### DESCRIPTION OF THE INVENTION

The present invention first relates to a cartridge 6 adapted to house at least one biological sample 58.

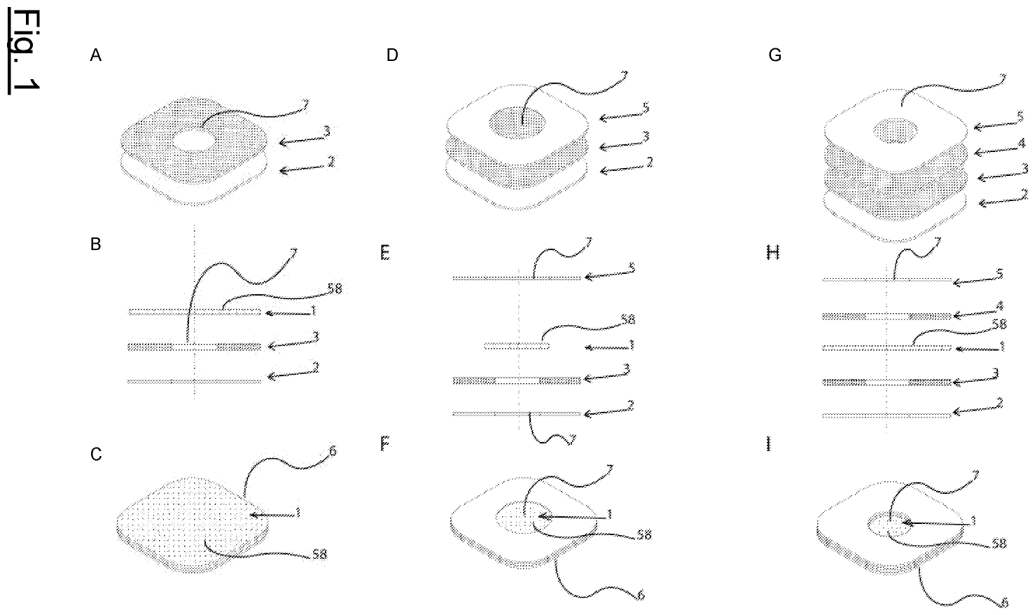

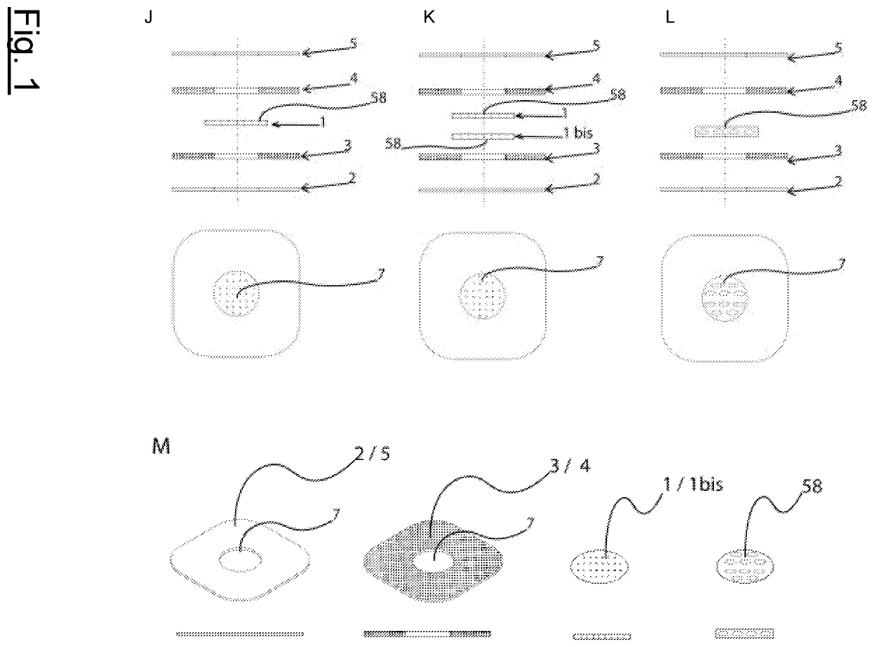

With reference to FIG. 1, said cartridge 6 comprises at least two overlapping layers where:
at least one layer consists of highly hydrophobic, inert, and biocompatible material: this layer is referred to in the present description with the term hydrophobic layer;
optionally, at least one layer consisting of a double-sided adhesive material.

In the embodiments not comprising said at least one layer of double-sided adhesive material, the adhesive function is obtained by chemical and/or physical bonding.

Preferably, said at least two overlapping layers have an almost square shape. Said at least two overlapping layers have at least one inner hole. When said two layers overlap each other to form said cartridge, said at least one inner hole on each of said layers causes said cartridge to in turn have at least one inner hole. In an embodiment, there is one said inner hole and it is placed in an almost central position on said cartridge 6.

Said cartridge 6 is adapted to house at least one biological sample 58, optionally supported on at least one membrane 1.

When housed in said cartridge 6, at least one portion of said at least one biological sample 58 occupies said inner hole, in whole or in part.

FIG. 1M shows the color reference used for the hydrophobic layers 2, 5, the double-sided adhesive material layers 3, 4, the membrane 1, 1bis, the self-supporting biological sample 58.

With reference to FIGS. 1A, 1B, 1C, in an embodiment said cartridge 6 comprises two overlapping layers 2, 3 of almost square shape:
a hydrophobic layer 2;
a layer 3 of double-sided adhesive material.

Said two overlapping layers 2, 3 have an inner hole 7.

In this embodiment, the biological sample 58 is housed on said layer 3 of double-sided adhesive material, supported on a membrane 1.

Said biological sample is housed in said cartridge such that a portion of said biological sample and/or of said membrane which supports it forms a further layer completely overlapping said layer 3 of double-sided adhesive material which holds it in place, the remaining portion of said biological sample and/or membrane which supports it going to occupy said at least one inner hole 7.

In a second embodiment (FIGS. 1D, 1E, 1F), said biological sample 58 is housed in said cartridge 6 overlapping said layer 3 of double-sided adhesive material such that said biological sample and/or said membrane which supports it only partially occupies said layer 3 of double-sided adhesive material which holds it in place. In this embodiment, a second hydrophobic layer 5 occupies the remaining portion of said layer 3 of double-sided adhesive material, so as not to expose the adhesive surface of the layer 3 to the outside.

In a third embodiment (FIGS. 1G, 1H, 1I), said cartridge 6 comprises four overlapping layers. Outside, FIG. 1G, there are two hydrophobic layers 2, 5. Closed between said two hydrophobic layers 2, 5, there are two layers 3, 4 of double-sided adhesive material. Said four overlapping layers 2, 3, 4, 5 have an inner hole 7. When said four layers overlap one another to form said cartridge, said inner hole 7 on each of said layers causes the cartridge itself to have an inner hole 7. At least one biological sample 58 is housed between said two layers 3, 4 of double-sided adhesive material (FIGS. 1H, 1I), optionally supported on a membrane 1.

Said at least one biological sample is housed in said cartridge such that a portion of said at least one biological sample 58 (FIG. 1L) and/or of said at least one membrane 1 which supports it (FIGS. 1J, 1K) forms a further layer between said two layers 3, 4 of double-sided adhesive material which hold it in place, the remaining portion of said biological sample and/or membrane which supports it going to occupy said at least one inner hole 7.

In an embodiment, with reference to FIG. 1J, said biological sample is supported on a membrane 1. By way of example, said biological sample 58 comprises cells plated on said membrane 1.

Alternatively, with reference to FIG. 1K, said at least one biological sample 58 is supported on two membranes, a first membrane 1 and a second membrane 1bis. By way of example, a first biological sample 58 rests on a first membrane 1 and a second biological sample 58 rests on a second membrane 1bis.

In a further embodiment, with reference to FIG. 1L, said biological sample 58 is self-supporting. In this embodiment, said biological sample 58 is a viable biological tissue, a decellularized biological tissue, a cellularized three-dimensional scaffold, a cellularized hydrogel. In this embodiment, said at least one membrane 1 is not required and said biological sample 58 is housed between said layers 3, 4 of double-sided adhesive material.

In an embodiment, said cartridge 6 houses two distinct biological samples 58, both of which are self-supporting. Said first self-supporting biological sample rests on a first layer of double-sided adhesive material and said second self-supporting biological sample rests on a second layer of double-sided adhesive material, so that said two biological samples are interfaced.

In an embodiment, said cartridge 6 houses two distinct biological samples 58, a first self-supporting biological sample and a second biological sample resting on said at least one membrane.

Secondly, the present invention relates to a fluidic device 200, 500 for static and/or dynamic biological cultures, comprising at least one cartridge 6 inserted into a support 20, 50.

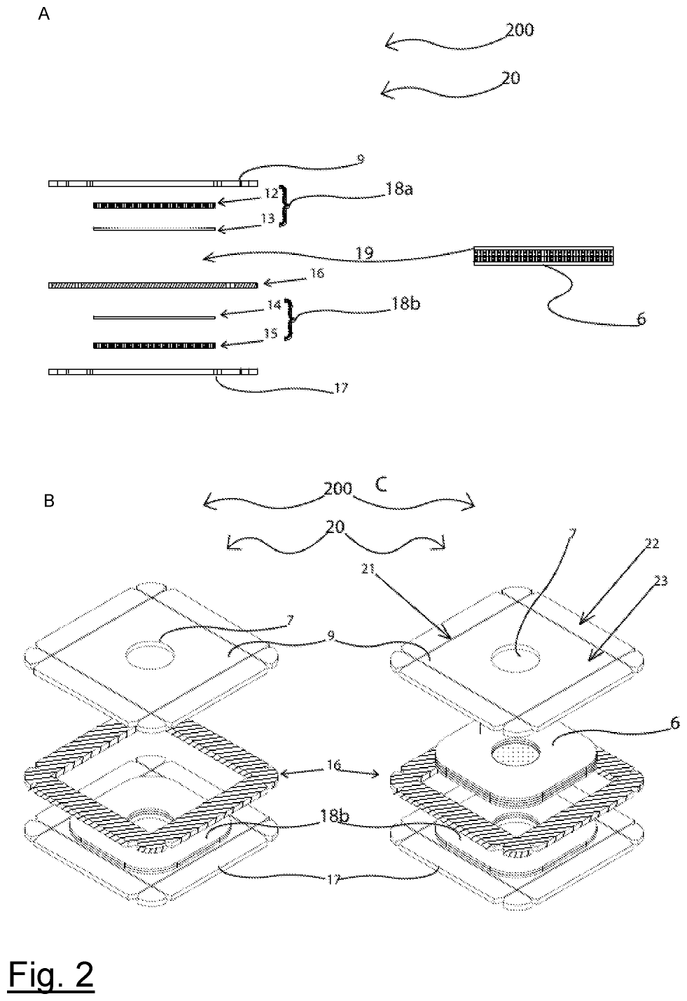

With reference to FIGS. 2, 3, 4, 5, 6, said fluidic device 200, 500 comprises:
at least one cartridge 6;
a support 20, 50, where said support comprises an upper pocket and, optionally, a lower pocket, said two pockets being enclosed between at least two rigid elements, at least one upper rigid element and at least one lower rigid element;
said at least one cartridge 6 being sandwiched between said upper pocket and said lower pocket, if this is present, or between said upper pocket and the lower rigid element if said lower pocket is not present;
characterized in that said rigid elements have weakening notches which identify an inner portion and an outer crown, where the area occupied by said at least one cartridge 6 is included in the area identified as the inner portion.

Said upper pocket comprises a layer of highly hydrophobic, inert, and biocompatible material, defined as a hydrophobic support layer and, optionally, a layer of double-sided adhesive material, defined as a double-sided adhesive support layer.

Said lower pocket comprises a hydrophobic support layer and, optionally, a double-sided adhesive support layer, said hydrophobic support layer facing said hydrophobic support layer of said upper pocket.

In the embodiments not comprising said at least one layer of double-sided adhesive material, the connection function between said layers is obtained by chemical and/or physical bonding.

In an embodiment, said support 20, 50 further comprises a double-sided adhesive frame 16, 32 having a seat 51 adapted to accommodate said at least one cartridge 6.

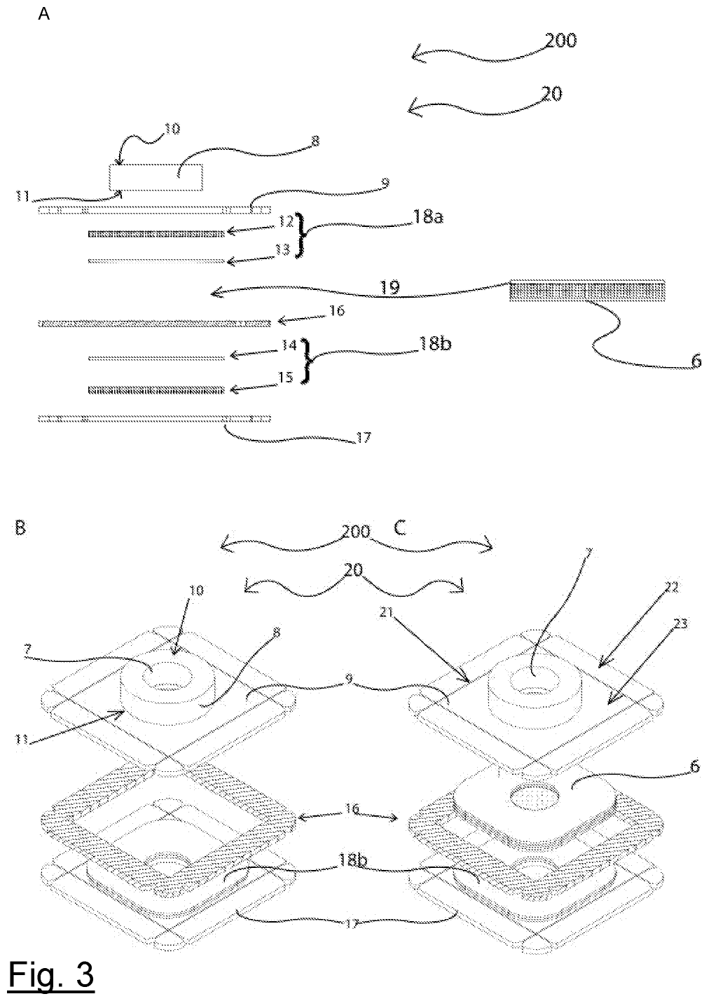

In an embodiment, with reference to FIGS. 2, 3, said fluidic device is a fluidic device 200 for static culture.

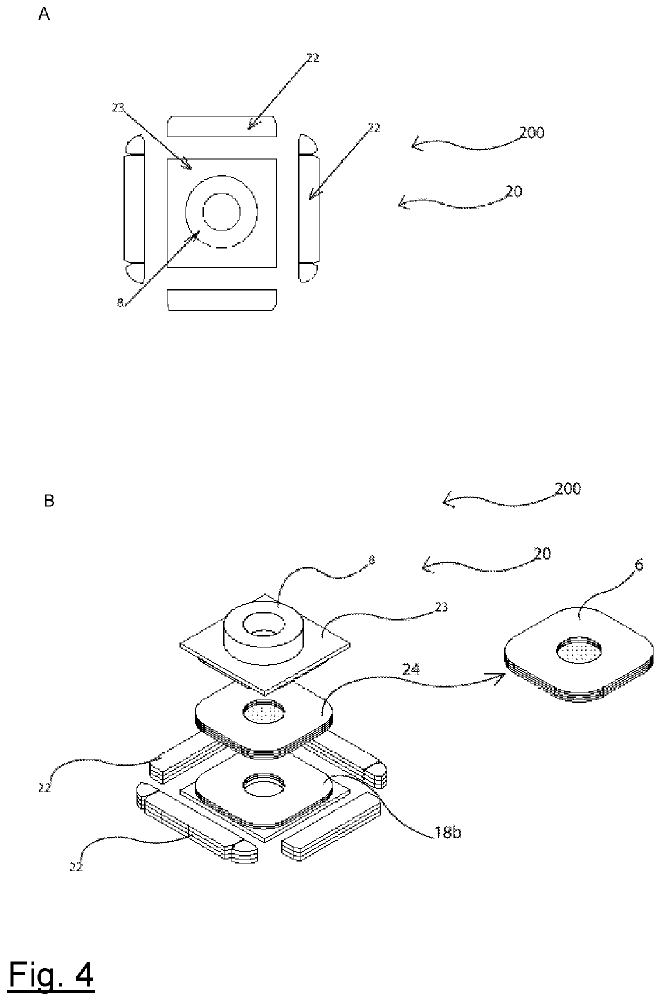

In a further embodiment, with reference to FIGS. 4, 5, 6, said fluidic device is a fluidic device 500 for dynamic culture.

In the fluidic device 200 for static culture, said support comprises:
Two rigid elements, where said rigid elements each have an inner face, towards the inside of the device, and an outer face, towards the outside of the device;
An upper pocket comprising a hydrophobic support layer and, optionally, a double-sided adhesive support layer;
Optionally, a lower pocket comprising a hydrophobic support layer and, optionally, a double-sided adhesive support layer, said hydrophobic support layer of said lower pocket facing said hydrophobic support layer of said upper pocket;
A double-sided adhesive frame adapted to accommodate said at least one cartridge 6.

In the embodiments not comprising one or more of said layers of double-sided adhesive material, the connection function between said layers is obtained by chemical and/or physical bonding.

In an embodiment, with reference to FIG. 2, said fluidic device 200 for static culture comprises a cartridge 6 and a support 20. Said support 20 comprises, proceeding from the outside towards the inside:
Two rigid elements 9, 17, where said rigid elements each have an inner face, towards the inside of the device, and an outer face, towards the outside of the device;
An upper pocket 18a comprising a layer in highly hydrophobic, inert, and biocompatible material, defined as a hydrophobic support layer 13, and a layer of double-sided adhesive material, defined as a double-sided adhesive support layer 12;
A lower pocket 18b comprising a hydrophobic support layer 14, and a double-sided adhesive support layer 15, said hydrophobic support layer 14 facing said hydrophobic support layer 13 of said upper pocket 18a;
A double-sided adhesive frame 16 having a seat 51 of a shape and size adapted to accommodate said at least one cartridge 6.

Said two upper 18a and lower 18b pockets have a shape almost similar to the shape of the layers embedded in said cartridge 6 and also have at least one inner hole 7. Said cartridge 6 is sandwiched between said upper and lower pockets 18a, 18b, where said hydrophobic support layers 13, 14 face inwards, i.e., towards said cartridge 6 and said two layers 12, 15 of double-sided adhesive support material face outwards, i.e., towards said two rigid elements, the upper rigid element 9 and the lower rigid element 17.

Preferably, said upper 9 and lower 17 rigid elements are made of a polymeric material which can be processed by conventional processes such as laser cutting, chip removal, milling, punching, molding, forming, casting, or they can be obtained by additive processes such as 3D printing, and have a controlled thickness.

Said support 20 is characterized in that said upper 9 and lower 17 rigid elements have weakening notches 21, as shown in FIGS. 2A, 2B, 2C. Said weakening notches 21 run along the perimeter of said upper 9 and lower 17 rigid elements and identify an inner portion 23 and an outer crown 22 in said upper 9 and lower 17 rigid elements. Advantageously, the area of said inner portion 23 is such as to comprise the area of said layers forming said cartridge 6.

In the embodiment in FIG. 2, said support 20 thus comprises said upper 18a and lower 18b pockets having at least one inner hole 7, sandwiched between said two upper 9 and lower 17 rigid elements. Said support 20 houses said cartridge 6 therein, between said upper 18a and lower 18b pockets, where said at least one inner hole 7 is at least one inner hole passing through said cartridge 6 and said pockets 18a, 18b.

In an embodiment, said upper 9 and lower 17 rigid elements have said at least one inner hole 7 as well. In this embodiment, said fluidic device 200 for static culture (FIGS. 2A, 2B, 2C), comprising said support 20 and said cartridge 6, is crossed by said at least one inner hole 7.

When said fluidic device 200 static culture is loaded with at least one biological sample 58, said at least one inner hole 7 is closed by said biological sample 58, optionally supported on at least one membrane 1.

In a further embodiment, shown in FIGS. 3A, 3B, 3C, said support 20 further comprises a well 8 having a distal end 10 and a proximal end 11, open at the proximal end 11, optionally open at the distal end 10, where said well 8 is sealingly positioned on the outer face of at least one of said two upper 9 and/or lower 17 rigid elements which also have said at least one inner hole 7.

Said well 8 is positioned such that the opening of said well is at said at least one inner hole 7 passing through said fluidic device 200.

In an embodiment, said well 8 is sealingly positioned on said at least one outer face of said upper 9 and/or lower 17 rigid element by means of a reversibly removable coupling.

In an embodiment, said well 8 is sealingly positioned on said at least one outer face of said upper 9 and/or lower 17 rigid element by means of a double-sided adhesive material.

In an embodiment, said well 8 is sealingly positioned on said at least one outer face of said upper 9 and/or lower 17 rigid element by means of an adhesive coupling, for example an adhesive coupling based on the surface features, obtained by the selection or the adjustment of the surface affinity of a material towards the material itself or another material.

In an embodiment, said well 8 is sealingly positioned on said at least one outer face of said upper 9 and/or lower 17 rigid element by means of a mechanical coupling, for example an interlocking coupling or an interference or press-fit coupling, or a conical coupling.

In a preferred embodiment, said well 8 is sealingly positioned on said at least one outer face of said upper 9 and/or lower 17 rigid element by means of magnetic components. In this embodiment, said at least one outer face of said upper and/or lower rigid element has a seat in which a magnetic component is housed, preferably said seat occupies the perimeter around said at least one inner hole 7. Similarly, a magnetic component is embedded in said open well 8. It is essential that said magnetic component is completely embedded in the material forming said rigid support and said well 8. In fact, if not perfectly embedded, said magnetic component could interfere with the biological sample inserted into said support 20.

In a further embodiment, where the distal end 10 of said well 8 is open, said well 8 is provided with a cap which is positioned on said distal end 10 so as to close it.

Said cartridge 6 interposed between said upper 18a and lower 18b pockets is received in said frame 16 so that said frame in double-sided adhesive material 16 allows the adhesion between said upper 9 and lower 17 rigid elements.

As shown in FIGS. 4A, 4B, said fluidic device 200 is easily opened by virtue of the controlled breaking of said support 20 along said weakening notches 21, so that said cartridge 6 contained therein can be recovered without being damaged.

In the fluidic device 500 for dynamic culture, said support is a multilayer which comprises, sandwiched and proceeding from the outside towards the inside:
two coverslips;
optionally, two layers of double-sided adhesive material;
two rigid elements;
an upper pocket comprising:
   optionally, a double-sided adhesive support layer;
   a hydrophobic support layer;
optionally, a lower pocket comprising:
   optionally, a double-sided adhesive support layer;
   a hydrophobic support layer;
a double-sided adhesive frame adapted to accommodate said at least one cartridge.

In the embodiments not comprising one or more of said layers of double-sided adhesive material, the connection function between said layers is obtained by chemical and/or physical bonding.

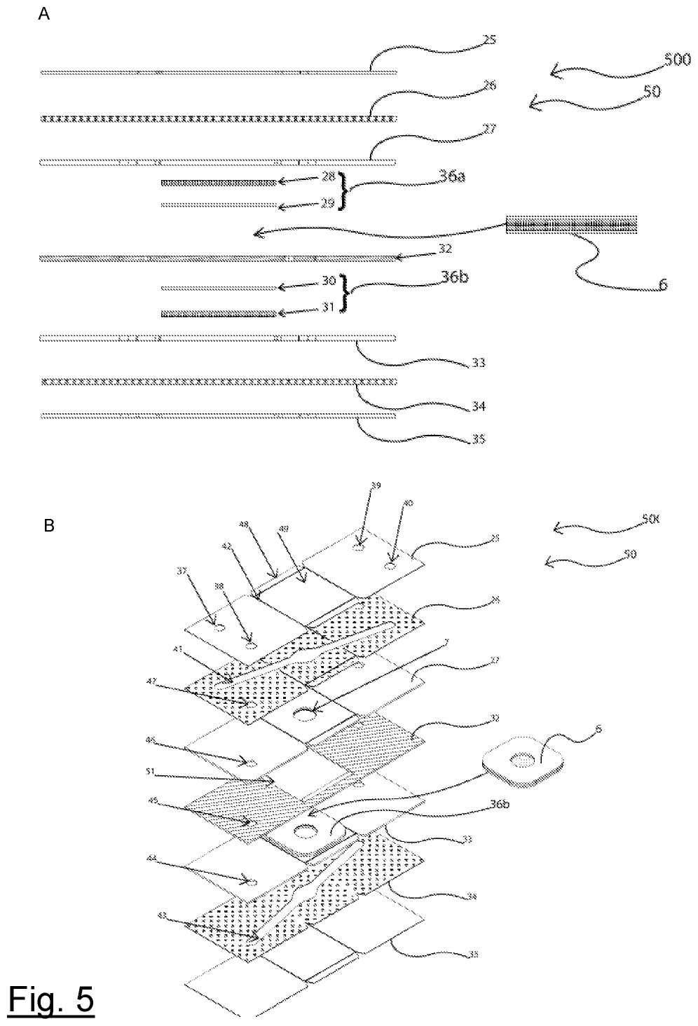

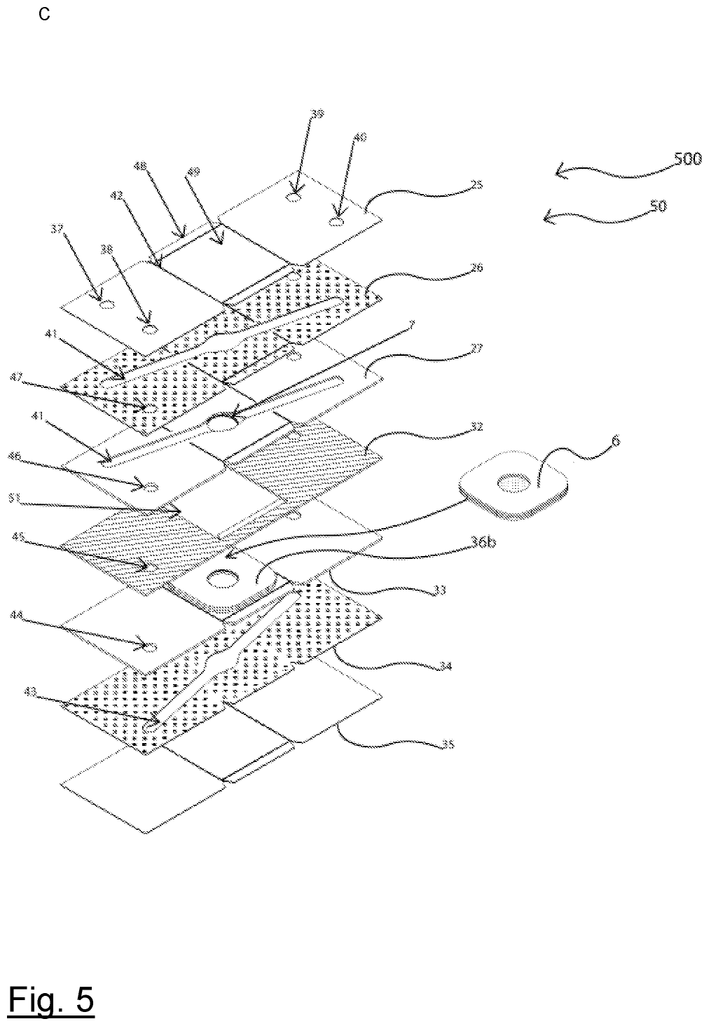

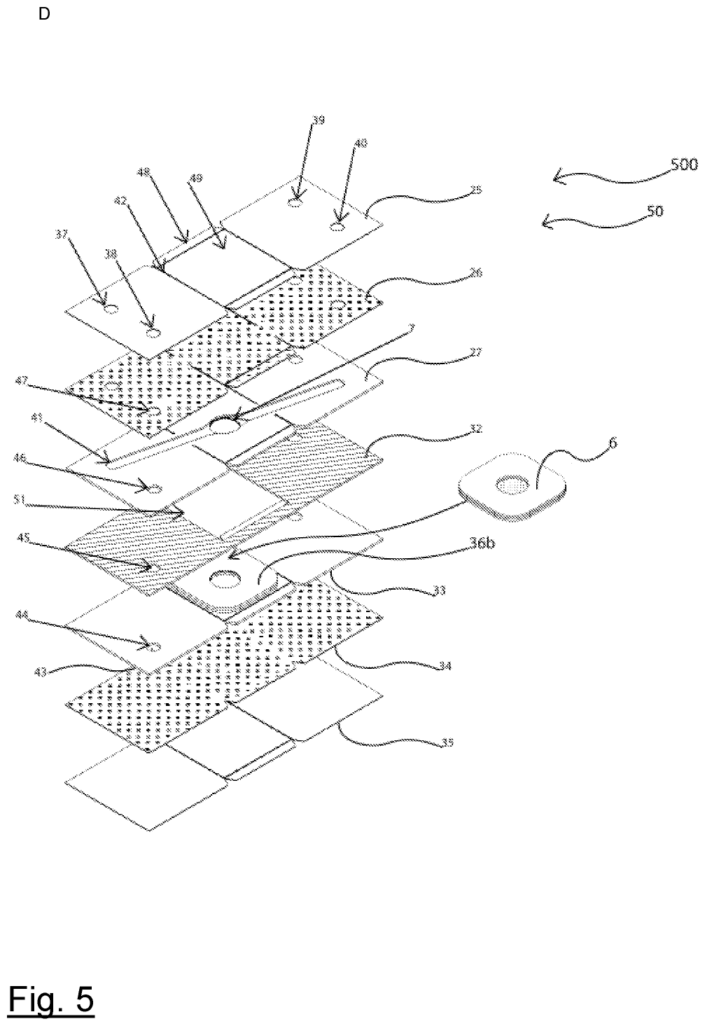

With reference to FIGS. 5A, 5B, 5C, 5D, said fluidic device 500 for dynamic culture comprises a cartridge 6 and a support 50.

Said support 50 is a multilayer which comprises, sandwiched and proceeding from the outside towards the inside:
two coverslips 25, 35;
two layers 26, 34 of double-sided adhesive material;
two rigid elements 27, 33;
an upper pocket 36a comprising:
   a double-sided adhesive layer 28;
   a layer of highly hydrophobic, inert, and biocompatible material, referred to as the hydrophobic support layer 29
a lower pocket 36b comprising:
   a double-sided adhesive layer 31;
   a hydrophobic support layer 30;
a double-sided adhesive frame 32 having a seat 51 of a shape and size adapted to accommodate said at least one cartridge 6.

Said rigid elements 27, 33 and said double-sided adhesive layers 26, 34 each have at least one inner hole 7.

Said coverslips 25 and 35 close the access to the sample from the outside when housed in said cartridge.

Said coverslips 25, 35, closing the access to the sample from the outside when housed in the cartridge, allow the maintenance and treatment of biological cultures in controlled physical and chemical conditions. In an embodiment, said coverslips are made of a material which is essentially impermeable to gases or other chemical agents present in the surrounding environment, so as to allow the progression of biological cultures under controlled chemical conditions, utilizing only the chemical-physical features of a microenvironment and/or a culture medium which comes into contact with the biological sample. Alternatively, said coverslips are made of a material adapted to utilize the diffusional balance of one or more chemical species with the surrounding environment. By way of example, said coverslips are made of a material which is highly permeable to gases such as oxygen and carbon dioxide, for example they are made of cellulose acetate butyrate or polydimethylsiloxane.

In this embodiment, said fluidic device preferably comprises a series of inlet-outlet accesses, which put said biological sample in communication with the external environment when housed in said cartridge. Said accesses face the outside of the fluidic device from the upper and/or lower surfaces and/or from the lateral edges of said fluidic device. There are 1, 2, 3, 4, 5, 6, 7, 8 or more accesses. In a particularly preferred embodiment, there are 2, 3, 4, 5, or 6. Said accesses are inlets and/or outlets. In an embodiment, said accesses are in fluidic connection with one another, for example, they are coupled two by two, i.e., an inlet access is in fluidic connection with an outlet access. Said fluidic connection is obtained through a channel, where said channel is obtained for example in the thickness of one or more of said double-sided adhesive layers and/or in the thickness of one or more of said rigid elements. By way of example, the embodiment in FIGS. 5A, 5B comprises four accesses, two lateral inlet and/or outlet 37, 38 and two contralateral inlet and/or outlet 39, 40 accesses. Said accesses face the outside of the fluidic device from the upper and/or lower surfaces of said device. Said inlet-outlet accesses are coupled together and connected by a first and/or a second channel 41 and/or 43, said first and/or second channels 41, 43 are obtained in the thickness of said double-sided adhesive layers 26 and 34, respectively. Said first and second channels put said at least one biological sample 58 occupying said at least one inner hole 7 in communication with the external environment. By way of example, in the fluidic device in the embodiment in FIGS. 5A, 5B, said accesses form two pairs, a first pair 37, 40, a second pair 38, 39. Said first pair 37, 40 accesses a first channel 41, obtained in said double-sided adhesive layer 26. Said second pair 38, 39 accesses a second channel 43, obtained in said double-sided adhesive layer 34. In this embodiment, said first channel 41 and said second channel 43 are in fluid communication with each other only through the at least one biological sample 58 occupying said at least one inner hole 7. In fact, said first 41 and second 43 channels both reach said at least one biological sample 58, said first channel from one side, said second channel from the opposite side. Therefore, the fluid communication between said first and second channels necessarily passes through said at least one biological sample.

These and other embodiments of the relative arrangement of the inlet-outlet accesses, the channels and the hole can be obtained by utilizing all the possible combinations of shape, size, position, orientation, and number of said cavities.

With reference to FIG. 5C, in a further embodiment said channels 41 and/or 43 are formed both in the thickness of said rigid elements 27 and/or 33, and in said double-sided adhesive layers 26 and/or 34.

In another embodiment, with reference to FIG. 5D, the channels 41 and/or 43 are obtained in the thickness of the rigid elements 27 and/or 33, occupying the entire thickness thereof or even only part of the thickness. In this embodiment, the stable adhesion between said coverslips 25 and/or 35 and said rigid elements 27 and/or 33 can alternatively be obtained, if deemed convenient, by chemical and/or physical bonding, thus avoiding the interposition of the double-sided adhesive layers 26 and/or 34.

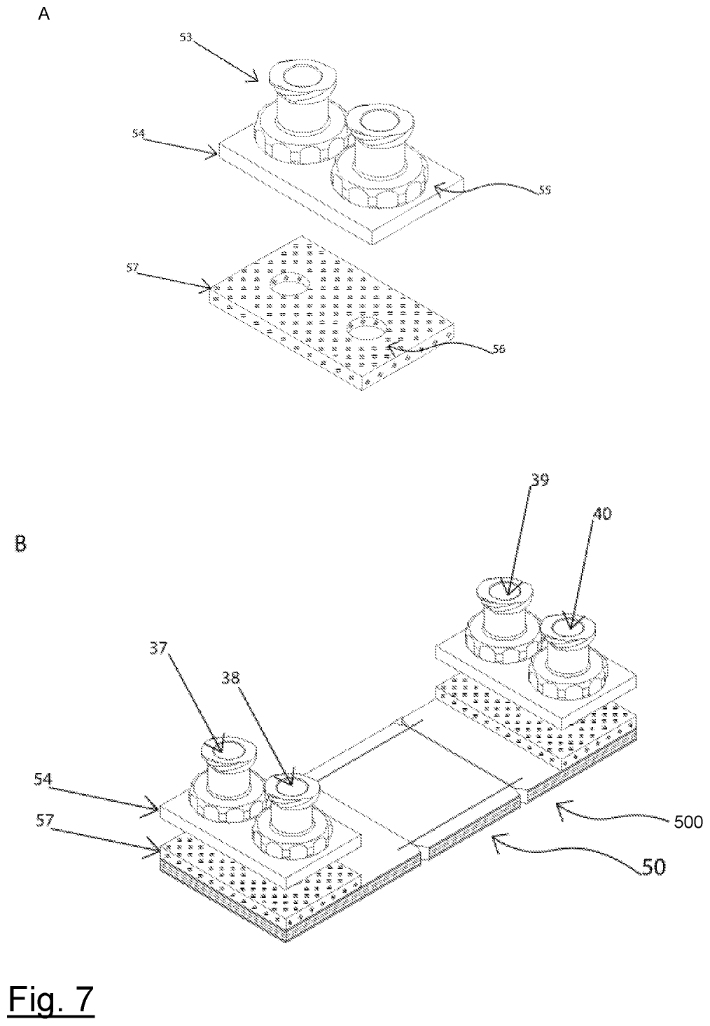

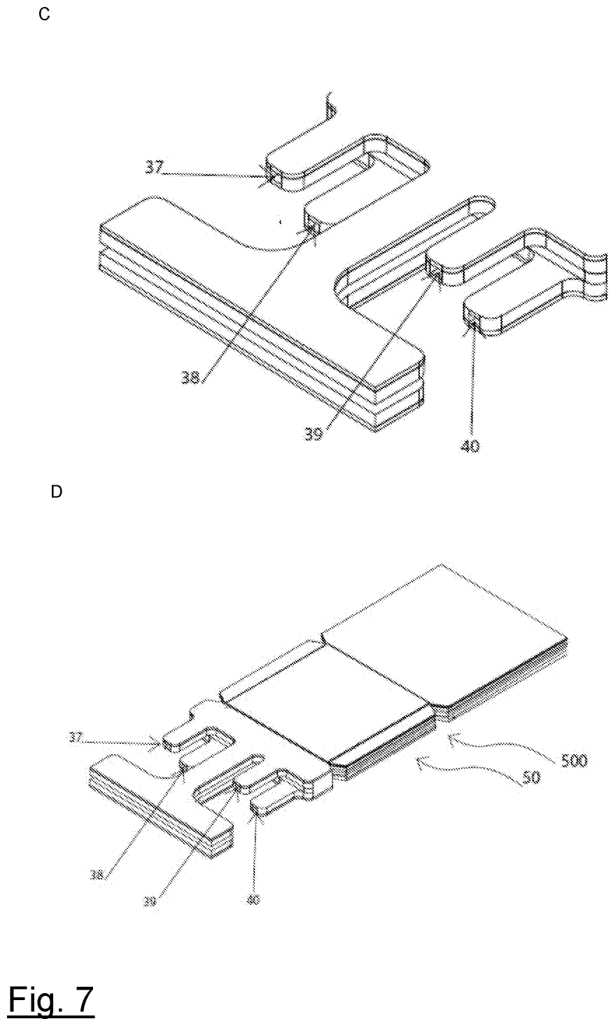

In a further embodiment, diagrammatically shown in FIGS. 7C, 7D said inlet-outlet accesses face the outside of the fluidic device from the lateral edges of the device itself. In this embodiment, said inlet-outlet accesses are essentially coplanar with the inner channels with which they communicate, where said channels are conveniently formed in the thickness of adhesive layers and/or of said rigid elements and/or of said coverslip.

The complex consisting of said inlet-outlet accesses and said channels, called access-channel complex, allows said biological sample housed in said cartridge to be put in communication with the external environment, allowing the implementation of different and multiple functions correlated with the development of a dynamic culture. Some of these functions are listed below, without claiming that this list is exhaustive of all the possible functions which can be obtained and/or of how those skilled in the art can combine similar or different functions for the purpose of the progression of a dynamic culture. Said accesses-channels complex can perform a first function of conducting fluid from the external environment towards the biological sample and/or, conducting fluid from the biological sample towards the external environment. For example, through the accesses-channels complex, culture medium loaded with cells can be conveyed towards the region designated to house the biological sample to seed said cells on a support membrane. For example, through the accesses-channels complex, fresh culture medium can be introduced into the system and/or exhausted culture medium can be removed, thus allowing the renewal of the culture microenvironment with which the biological sample interacts. For example, through the accesses-channels complex, a substance in solution or suspension in the culture medium at the desired concentration can be conveyed by convective transport towards the biological sample, for example to condition the microenvironment. For example, through the accesses-channels complex, the culture medium which is part of the culture microenvironment with which the biological sample interacts can be conveyed to the outside, for example to analyze the features thereof or to extract substances produced by the biological activity of the biological sample. For example, through the accesses-channels complex, a culture medium loaded with particles, or with cells, or with biological agents can be conveyed towards the biological sample to study the interaction between such particles, cells, biological agents with the biological sample.

Said accesses-channels complex can perform the further function of conducting signals from the external environment towards the biological sample and/or, conducting signals from the biological sample towards the external environment. For example, an electrical signal can be conducted through the accesses-channels complex. A signal of this nature travels through the channels if suitable means capable of conducting such a signal are arranged along said channels, for example an electrical conducting fluid, such as the culture medium itself, or another conductive or semiconductive material which fills the channel itself, also partially, for example one or more metal wires. For example, a light signal or a similar electromagnetic signal which does not fall within the visible spectrum, for example an infrared or ultraviolet signal, can be conducted through the accesses-channels complex. A signal of this nature travels through the channels if suitable means capable of conducting such a signal are arranged along said channels by virtue of the effect utilized by the optical fibers. For example, a vibrational signal, for example a sound or ultrasound signal, can be conducted through the accesses-channels complex. A signal of this nature travels through the channels if suitable means capable of conducting such a signal are arranged along said channels, for example a fluid having an acoustic impedance significantly different from the acoustic impedance of the materials forming the fluidic device.

Said accesses-channels complex can perform the further function of inducing mechanical stresses on the biological sample. For example, a pressure stress can be applied to the biological sample through the accesses-channels complex. Such a stress can be obtained by pressurizing the fluid contained in the accesses-channels complex. For example, a surface shear stress, otherwise known as wall shear stress, can be applied to the biological sample through the accesses-channels complex. Such a stress can be obtained by moving the fluid contained in the accesses-channels complex according to a law of motion suitably designed to induce a controlled tangential stress on the surface of the biological sample, for example due to the viscosity of the fluid.

These and other characteristic functions of a dynamic culture are practicable in a different manner on the two faces of the biological sample, by virtue of the distinct arrangement of the accesses-channels complex interacting with the upper face of the biological sample with respect to the accesses-channels complex interacting with the lower face of the biological sample.

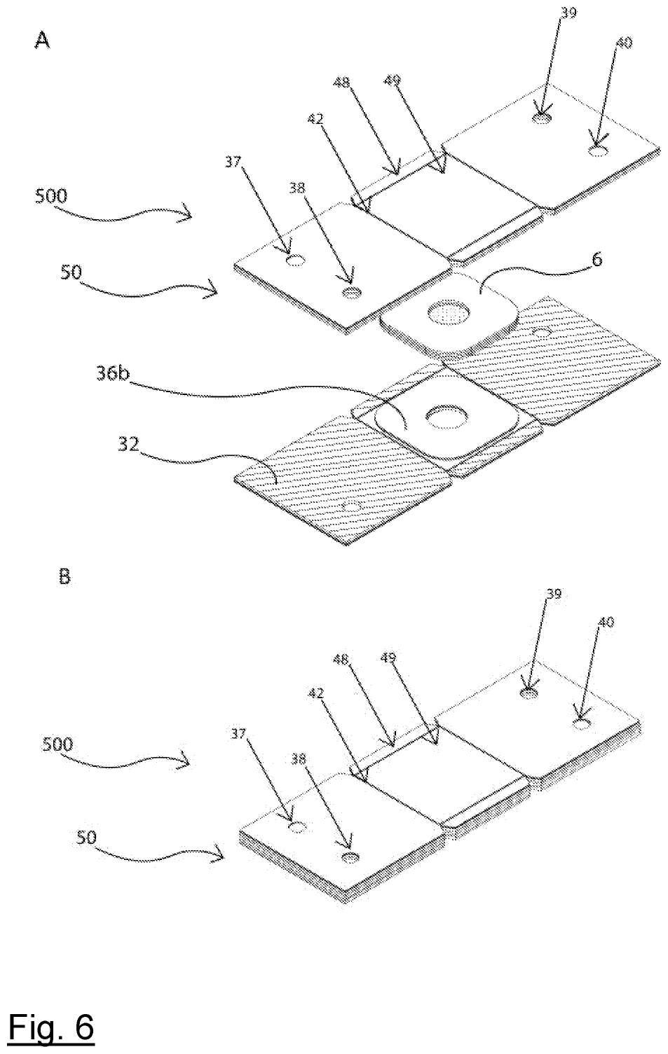

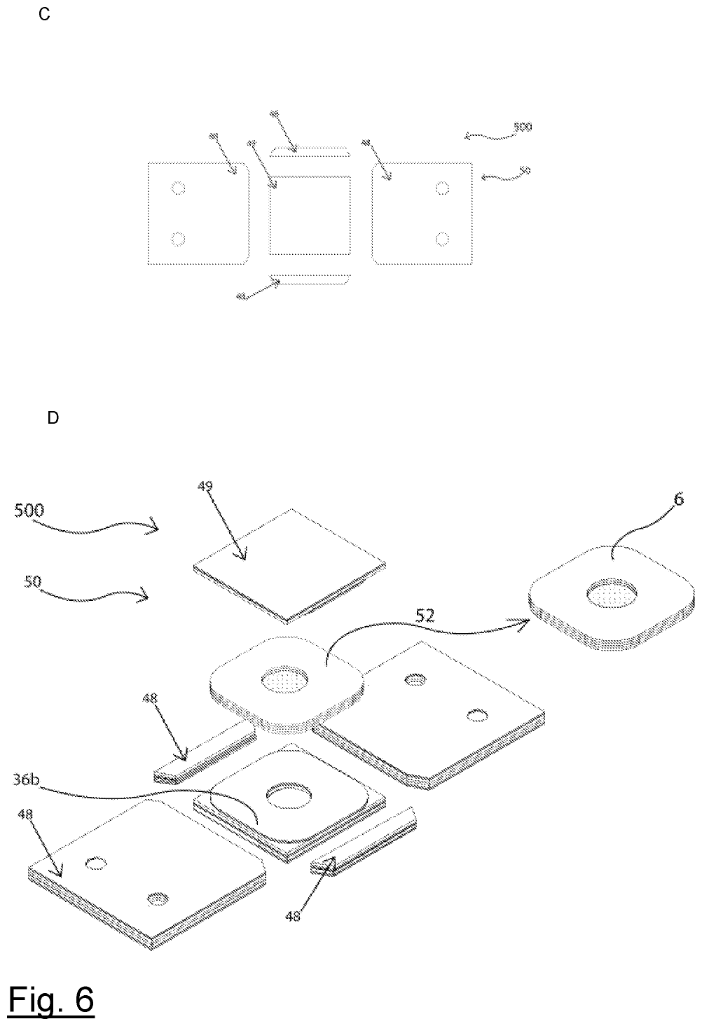

The supports in embodiment 50 are characterized by weakening notches 42, as shown in FIGS. 6A, 6B. Said weakening notches 42 are on said coverslips 25, 35, on said double-sided adhesive layers 26, 34, on said rigid elements 27, 33, and identify an inner portion 49 and an outer crown 48 in said support. Advantageously, the area of said inner portion 49 is such as to encompass the area which accommodates said at least one cartridge 6. As shown in FIG. 6C, said support 50 is easily opened by virtue of the controlled breaking of said support 50 along said weakening notches 42. At the moment of the breaking, as diagrammatically shown in FIGS. 6C, 6D, the at least one cartridge 6 is recovered without it and the at least one biological sample 58 housed therein being damaged, without the need for manipulations which alter the sterility thereof.

In fact, said weakening notches allow the controlled breaking, along predetermined lines, to be carried out manually or with the aid of tools which are suitable for the purpose, such as laboratory tweezers, commercially available breaking pliers or other accessory tools specially made for the purpose.

In an embodiment, said fluidic device (200, 500) has a hydraulic connection which makes use of connectors, where said connectors are conveniently sealingly positioned outside said fluidic device, at said inlet and/or outlet (37, 38, 39, 40). By way of example, said connectors can be standard connectors, such as Luer-type connectors.

In an embodiment, said connectors are sealingly positioned on at least one outer face of said support 20, 50 by means of a reversible coupling.

In a preferred embodiment, said connectors are sealingly positioned on at least one outer face of said support 20, 50 by means of magnetic components. For example, with reference to FIG. 7A, Luer connectors 53 are positioned on a polymeric support 54. A magnetic ring 55 is embedded inside said polymeric support 54. In this embodiment, said polymeric support 54 is conveniently positioned on a polymeric support 57, inside which a further magnetic ring 56 is embedded. FIG. 7B shows how said polymeric support 57 is irreversibly constrained on said support 50, bringing said Luer connectors 53 to said inlet and/or outlet ports 37, 38, 39, 40 (FIG. 7B).

In an embodiment, at least one of said fluidic devices 200, 500 is conveniently housed in a support station 65, forming a fluidic devices-support station complex 600.

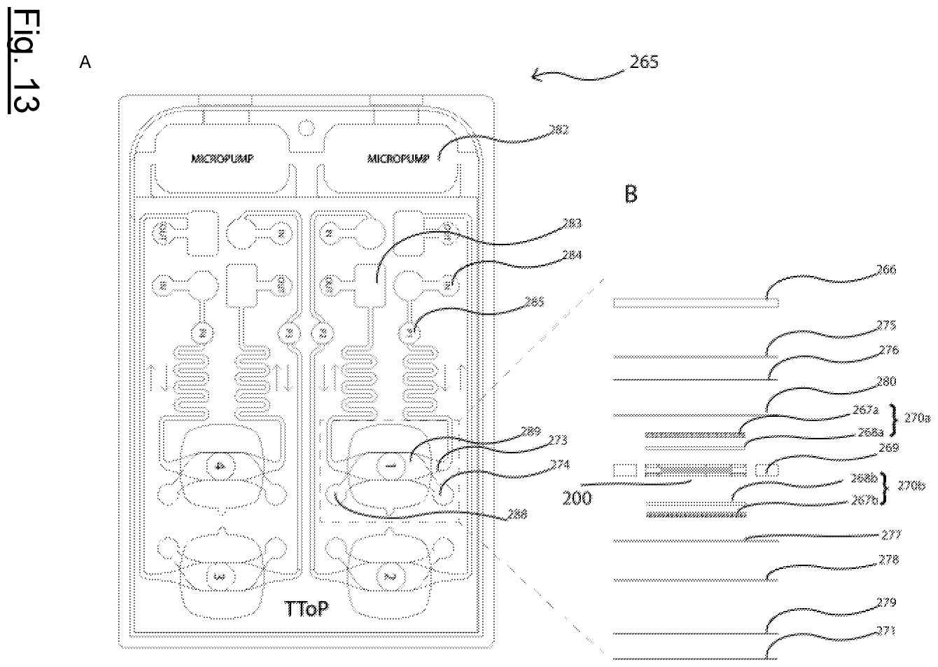

With reference to FIG. 12, said support station 65 is a multilayer comprising, sandwiched and proceeding from the outside towards the inside:
Two rigid elements 66, 71, where said rigid elements, which are optionally coverslips, each have an inner face, towards the inside of the support station, and an outer face, towards the outside of the support station;
A frame 69, interposed between said two rigid elements 66, 71, having a plurality of seats 86 each of a shape and size adapted to accommodate one of said fluidic devices 200, 500;
   and, at each of said plurality of seats 86,
an upper pocket 70a housed between said seat 86 and said rigid element 66, comprising a hydrophobic support layer 68a and, optionally, a double-sided adhesive support layer 67a;
optionally, a lower pocket 70b housed between said seat 86 and said rigid element 71 comprising a hydrophobic support layer 68b and, optionally, a double-sided adhesive support layer 67b,
   in which said hydrophobic support layers 68a and 68b face said seat 86.

In the embodiments not comprising one or more of said layers 67a, 67b of double-sided adhesive material, the connection function between said layers is obtained by chemical and/or physical bonding.

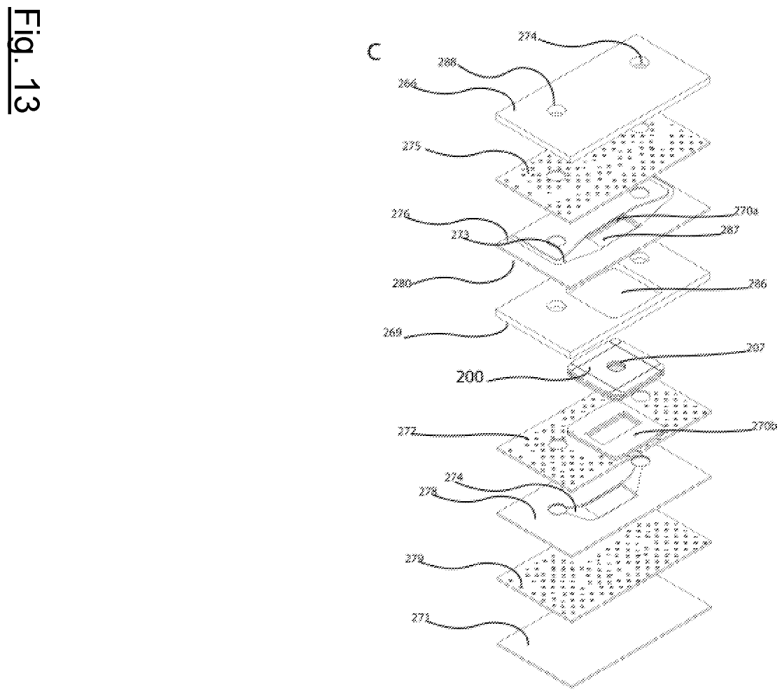

In a further embodiment, with reference to FIG. 13B, FIG. 13C said support station 265 is a multilayer comprising, sandwiched and proceeding from the outside towards the inside:
Two rigid elements 266, 271, which optionally are coverslips;
Two layers 275, 279 of double-sided adhesive material;
Two layers 276, 278 of rigid material;
Two layers 277, 280 of double-sided adhesive material;
A frame 269, interposed between said two layers 276, 278 of rigid material, having a plurality of seats 286, each of a shape and size adapted to accommodate one of said fluidic devices 200;
and, at each of said plurality of seats 286,
An upper pocket 270a, housed between said seat 286 and said layer 276 of rigid material comprising:
   optionally, a double-sided adhesive support layer 267a;
   a layer of highly hydrophobic, inert, and biocompatible material, which is a hydrophobic support layer 268a;
Optionally, a lower pocket 270b, housed between said seat 286 and said layer 277 of rigid material comprising:
   optionally, a double-sided adhesive support layer 267b;
   a layer of highly hydrophobic, inert, and biocompatible material, which is a hydrophobic support layer 268b;

Said layers 276, 278 of rigid material and said double-sided adhesive layers 277, 280 each have at least one inner hole 287.

In an embodiment, said coverslips 66, 71, 266, 271 are made as described for the coverslips 25, 35, i.e., in a material essentially impermeable to gases or other chemical agents present in the surrounding environment. The device-station complex 600 preferably comprises a series of inlet-outlet accesses, which put said biological sample in communication with the external environment when housed in said fluidic system 200.

As diagrammatically shown in FIG. 12A, said accesses face the outside of the fluidic device from the upper and/or lower surfaces and/or from the lateral edges of said device-station complex 600. There are 1, 2, 3, 4, 5, 6, 7, 8 or more accesses. Said accesses are inlet and/or outlet accesses and allow a fluidic connection with the external environment. Said fluidic connection is obtained through a channel, where said channel is obtained for example in the thickness of one or more of said double-sided adhesive layers and/or in the thickness of one or more of said rigid elements.

By way of example, the embodiment in FIG. 13A comprises four accesses, two inlet and/or outlet 274, 288, one inlet 284 and one outlet 283. Said inlet-outlet accesses are coupled to one another and connected by a first channel 289 and a second channel 273, respectively, where said first and/or second channels 289 and 273 are obtained in the thickness of said double-sided adhesive layers 275, 279 and/or in the thickness of said rigid elements 276, 278. Said first and second channels put said at least one biological sample (not shown) which, inserted into said cartridge housed in said fluidic device 200 placed in said complex 600, occupies said at least one inner hole 207, in communication with the external environment. In this embodiment, said first channel 289 and said second channel 273 are in fluid communication with each other only through the at least one biological sample. Said first 273 and second 289 channels both reach said at least one biological sample, said first channel from one face, said second channel from the opposite face. Therefore, the fluid communication between said first and second channels necessarily passes through said at least one biological sample. In FIG. 13A, the complex houses four fluidic devices, indicated with the circled numbers 1, 2, 3, 4. The movement of the fluids inside said channels is conveniently allowed by micropumps 282. In the embodiment in FIG. 13A, said at least one micropump 282 is housed in said complex 600.

The embodiment comprising a support station advantageously minimizes the manual procedures to be carried out by the user, making a simple, “plug and play” system available. Furthermore, with reference to the embodiment described in FIG. 13, the solution allows:
having optical access to at least one surface of the biological sample;
ensuring an easy assembly of the fluidic system 200, 500 inside the support station;
ensuring the maintenance of the system compartmentalization and thus allowing a controlled and optionally different treatment for each of the two exposed surfaces of the biological sample;
maintaining sterility during use, thus avoiding contamination;
being able to stimulate a multiplicity of fluidic systems 200, 500 independently.

These and other embodiments of the relative arrangement of the inlet-outlet accesses, the channels and the hole can be obtained by utilizing all the possible combinations of shape, size, position, orientation, and number of said cavities. The present invention further relates to a method for manufacturing the fluidic device according to the present invention.

In a first embodiment, a cartridge 6 is made available comprising at least one membrane. Said cartridge is then housed in a support 20, 50. Said biological sample is placed on said membrane. According to this embodiment, the membrane is previously stretched in the cartridge, therefore said procedure has no impact on the preservation and placement of the biological sample.

In a second embodiment, a cartridge 6 is made available which does or does not comprise said at least one membrane, where said cartridge is previously loaded with said biological sample, after which said cartridge 6 is housed in the support 20, 50. This alternative embodiment where the biological sample is supported on a membrane becomes the only possible embodiment where the biological sample is not membrane-supported. In this second embodiment, during the assembly step of said cartridge, said biological sample is inserted into said cartridge, so that it is held in place between said two layers 3, 4 of double-sided adhesive material.

The fluidic device according to the present invention, in the embodiments thereof, is conveniently used with static and/or dynamic biological cultures, in mono or bicompartmental models.

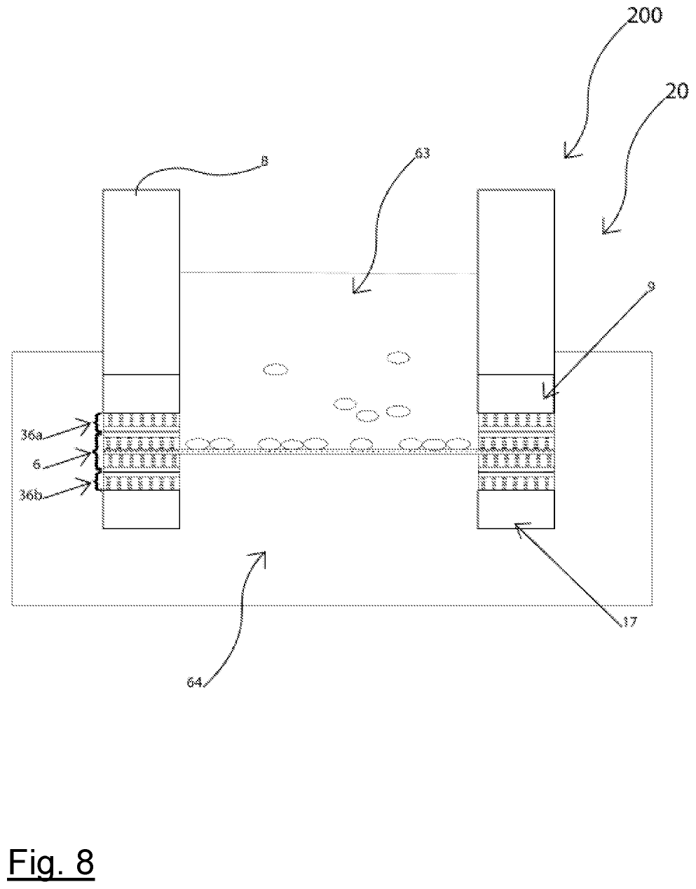

By way of example, FIG. 8 diagrammatically shows the use of the fluidic device according to an embodiment in a static culture in a bicompartmental model. In this use example, the fluidic device for static culture is placed inside a traditional system, which is a Petri dish or a well of a multi-well plate. FIG. 8 shows the two chambers which are created, an upper chamber 63, defined by said well 8 and a lower chamber 64 in continuity with the inner volume of the traditional system in which the fluidic device for static culture is inserted. Said upper chamber 63 is filled with liquid medium by means of a suitable instrument, by way of example said instrument can be an automatic pipettor. Said lower chamber 64 is filled with liquid medium by means of a suitable instrument, by way of example said instrument can be an automatic pipettor. In this embodiment, said chambers 63, 64 are not in fluid communication with each other, except through the sample loaded on said fluidic device.

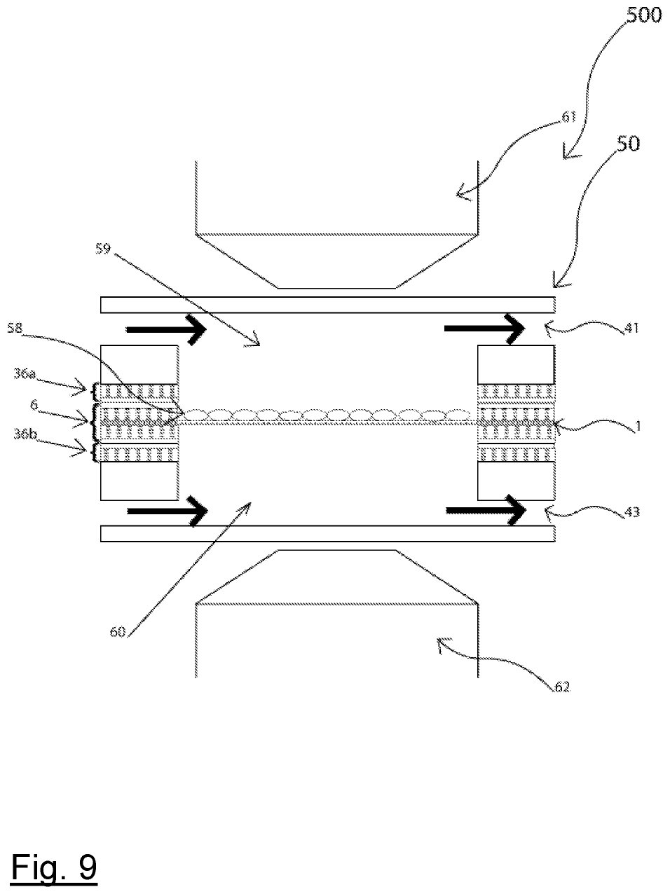

By way of example, FIG. 9 diagrammatically shows the use of the fluidic device according to the present invention in a dynamic culture in a two-compartment model. The biological sample 58, which in the specific case comprises cells, is cultured on a membrane 1 which is for example a microporous membrane positioned inside said cartridge 6, said cartridge 6 being housed in a support 50. The two chambers which are created are shown, an upper chamber 59 and a lower chamber 60. Said upper chamber 59 is crossed by the flow of liquid medium which passes through said first channel 41. Said lower chamber 60 is crossed by the flow of liquid medium which passes through said second channel 43.

Said fluidic device can be suitably positioned and analyzed on both an upright and an inverted microscope, depicted in FIG. 9 by lenses 61, 62.

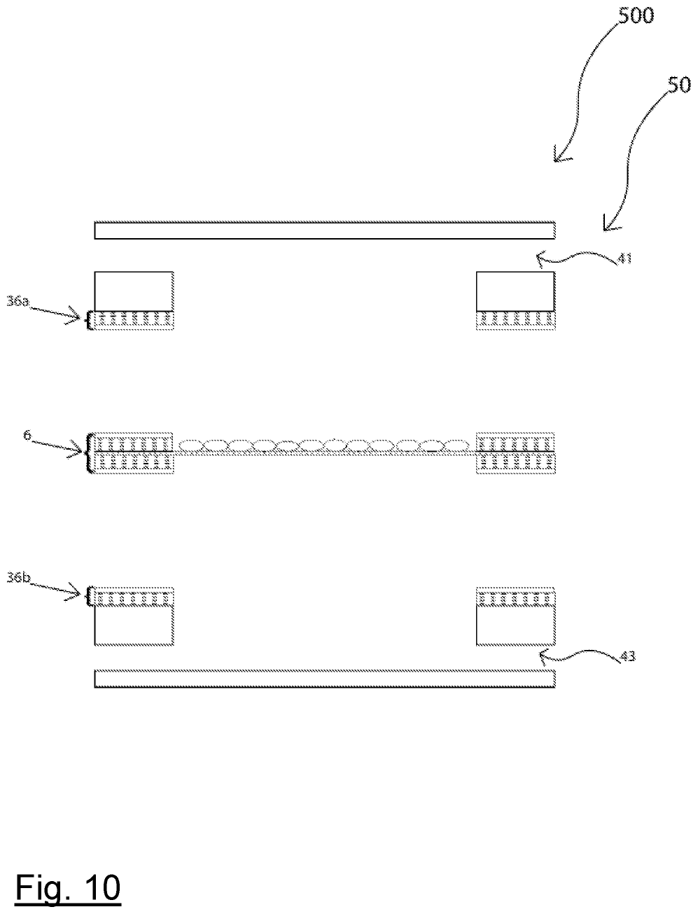

FIG. 10 shows the same fluidic device as in FIG. 9, after it has been opened following the weakening notches 42, so as to open said upper chamber and lower chamber towards the outside, allowing the recovery of said cartridge 6 containing the biological sample 58 for subsequent investigations.

The fluidic device according to the present invention, in the various embodiments thereof, has been successfully used in biological cultures, as better highlighted in the experimental section.

The following example has the sole purpose of better specifying the present invention, it is not to be understood as limiting it in any way, the scope of protection of which is defined by the claims.

### Example of a Cell Culture Experiment

The fluidic device 200 for static culture in the embodiment shown in FIG. 3 was used to conduct cell culture experiments. Intestinal epithelial cells, Caco-2 cells (continuous line of heterogeneous cells from colorectal adenocarcinoma, ATCC® HTB-37™), EA.hy926 endothelial cells (hybrid line obtained from stabilization of primary endothelial cells of the umbilical cord vein by fusion with A549/8 carcinoma cells, ATCC® CRL-2922™), and primary smooth muscle cells obtained from human internal mammary artery fragments (IMASMC, surgical waste from patients undergoing coronary artery bypass grafting) were used to check the system functionality, and in particular of the cartridge 6 and the static support 20.

The intestinal epithelial cells (Caco-2), endothelial cells (EA.hy926) and smooth muscle cells (IMASMC) were seeded and cultured on a membrane 1 sterilized with ethylene oxide, or pretreated with an alcohol-based solution to remove possible residual toxic elements present on the membrane itself, then kept in a hydrated state until seeding. The cell types tested are recognized as models and widely used as “gold standard” in biology studies in specific fields of application, i.e., in the study of the intestinal barrier, the endothelial barrier of the vascular lumen and the arterial wall. They were thus used as examples to demonstrate i) the suitability of the system for use in cell cultures and ii) the versatility of the system of the invention.

For the three cell types, we proceeded as follows: before inserting the system, an adequate amount of culture medium was introduced into the wells of a multi-well plate. The system was then inserted, taking care not to trap air under the membrane, then the cells were seeded on the pre-treated membrane 1 already inserted into the cartridge 6, in turn inserted into the static support 20, assembled and inserted into a well of the multi-well plate. The culture medium was also added to the well 8 where the cells were seeded, integrated in the support, to condition the compartment containing them.

The fluidic device loaded with the biological sample (cells supported on a membrane) was then kept in culture in a standard incubator at 37° C. and 5% CO₂ and verified every 2-3 culture days by microscopy until cell confluence was reached. The experiment with Caco-2 lasted at least 15 days, while the experiment with EA.hy926 lasted at least 8 days and at most 3 months.

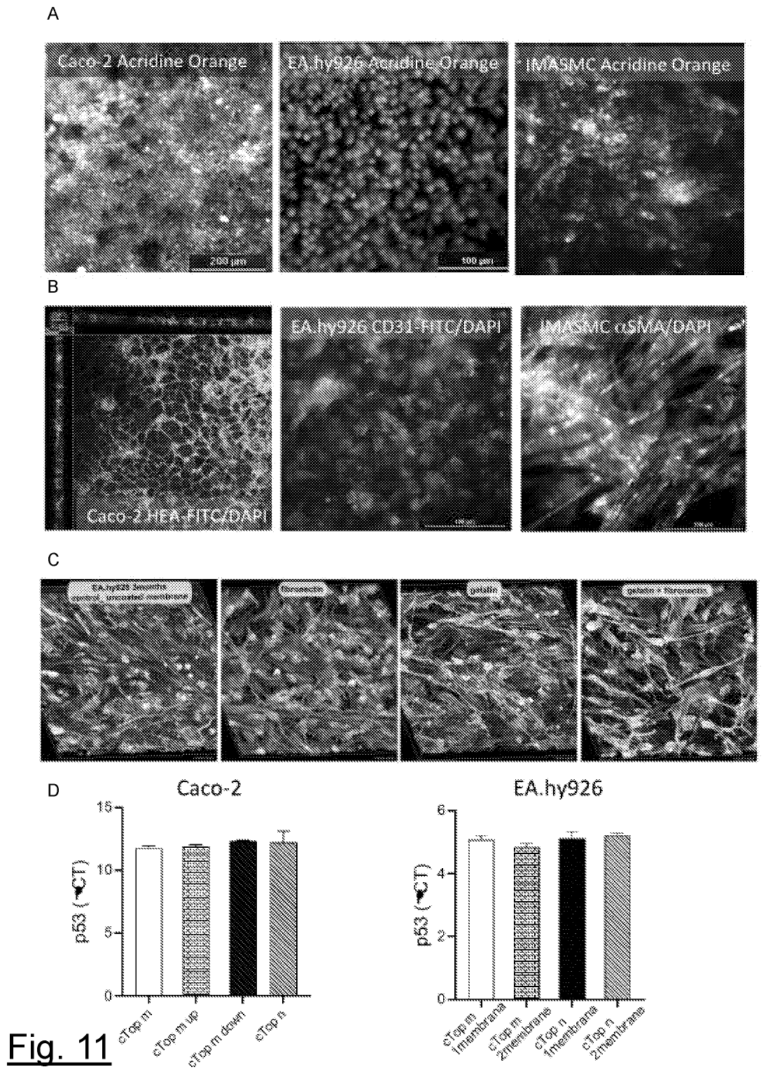

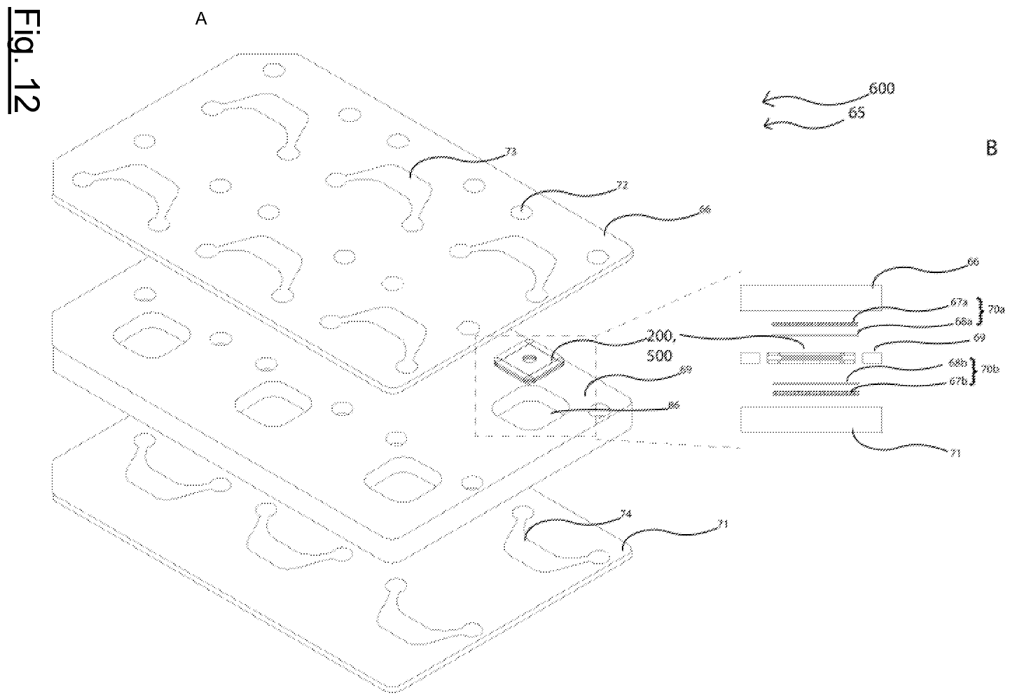

During culture, the viability, confluence, and homogeneity of the cell monolayer distribution within the fluidic device were periodically verified using Acridine Orange (code 318337, Sigma-Aldrich, St. Louis, Missouri, United States), a metachromatic nuclear intercalator compatible with cell viability, visible in fluorescence, which stains living cells. FIG. 11A shows the images obtained with Acridine Orange for the Caco-2 (left) and EA.hy926 (center) cells inserted into the fluidic device 200 for static culture, demonstrating the adequacy of the fluidic device according to the present invention for live imaging observations. With Acridine Orange, the sub-confluent IMASMC cells showed the typical in vitro “hills and valleys” distribution of smooth muscle cells of the vessels, thus confirming the adequacy of the device according to the present invention in allowing the morphological preservation of the cells during culture (FIG. 11A, right).

At the end of the culture the fluidic device 200 for static culture was opened, using the controlled breaking mechanism aimed at the release of the sample, as shown in FIG. 4, and the cartridge 6 containing the biological sample 58 was withdrawn intact from the static support 20 without the need to manipulate or alter the biological sample 58 to extract it.

Some of the membrane-supported biological samples extracted from the fluidic device were used to perform specific post fixation staining to evaluate cell differentiation. A counter-staining of the nuclei with a fluorescent nuclear intercalator, DAPI (code D9542, Sigma-Aldrich, St. Louis, Missouri, United States) was always added to the immunofluorescence stains. Other samples were instead processed for the Real Time Polymerase Chain Reaction (qRT-PCR) quantitative analysis.

Human epithelial antigen (HEA) expression and distribution was evaluated in Caco-2, using an anti-HEA monoclonal antibody (clone Ber-EP4, DAKO, Glostrup, Denmark) directly conjugated to fluorochrome FITC (fluorescein isothiocyanate). The antibody specifically recognizes a trans-membrane glycoprotein which mediates adhesion between epithelial cells, also known as EpCAM (FIG. 11B, left). The image obtained with confocal microscopy shows that the cells are arranged in a narrow monolayer and have the “brick” morphology typical of differentiated epithelial cells. The HEA marker highlights the location of cell junctions which supports the successful polarization of the Caco-2 cells.

The phenotype of the EA.hy926 cells was verified by staining the cells with an anti-CD31/PECAM1 monoclonal antibody (clone JC70A, DAKO, Glostrup, Denmark), an endothelial cell surface protein which mediates platelet binding. A secondary anti-mouse-IgG-AlexaFluor488 antibody (Catalog #A-11001, Invitrogen—Molecular Probes, Eugene, Oregon, USA) was used for detection. The images in FIG. 11B (center) obtained with a fluorescence microscope show CD31 positivity. The IMASMC cell phenotype was visualized by means of antibodies which recognize specific molecules of the smooth muscle cell cytoskeleton: the alpha isoform of smooth muscle actin (FIG. 11B, right). These verifications thus support the preservation of the phenotype of the cells grown in the fluidic device 200 for static culture.

Some cellular samples of Caco-2 and EA.hy926 extracted from the support 20 at the end of the culture were processed to perform qRT-PCR. The cells were detached from the membrane 1 by applying a standard enzymatic procedure with trypsin. RNA was isolated from the cells, back-transcribed to cDNA, then the cDNA was used for gene expression analysis by qRT-PCR. Purely as a feasibility check, for both cell lines examined, the gene expression of the transcription factor TP53 was evaluated using a specific probe conjugated to a reporter which emits fluorescence (Hs01034249_m1; Applied Biosystems, Foster City, CA, USA), which regulates the cell cycle and encodes the protein p53 (tumor protein 53) ubiquitously expressed by cells. FIG. 11C shows the expression of the gene TP53 in Caco-2 epithelial cells (left) and in EA.hy926 endothelial cells (right). The biological samples analyzed come from static devices with different features:
devices with standard well (cTop n), i.e., the fluidic device comprises a support with well sealingly positioned by means of a double-sided adhesive material;
devices with magnetic well (cTop m), i.e., the fluidic device comprises a support with well sealingly positioned by means of magnetic components;
presence of two membranes to allow co-culture, the two membranes of which were analyzed separately (cTop up, cTop down);
use of one or two pooled samples to perform the analysis (cTop 1 membrane, cTop 2 membranes).

The results obtained do not show quantitative differences for the different conditions explored, further demonstrating that the magnetic closure does not significantly influence the cell status.

1. A cartridge adapted to house at least one biological sample therein, wherein said cartridge comprises at least two overlapping layers, wherein said overlapping layers consist of:
at least one layer consisting of highly hydrophobic, inert, and biocompatible material, with contact angle Θ_{c}≥90°, hydrophobic layer;
optionally, at least one layer of double-sided adhesive material;
wherein, in the absence of said at least one layer of double-sided adhesive material, said overlapping layers are connected together by chemical and/or physical bonding;
wherein each of said overlapping layers has at least one inner hole and said at least one inner hole is pervious when said layers overlap one another;
wherein said at least one inner hole is closed by said at least one biological sample, where loaded in said cartridge.

2. The cartridge according to claim 1, further comprising at least one membrane adapted to support said at least one biological sample.

3. The cartridge according to claim 1, comprising at least 4 overlapping layers, wherein two hydrophobic layers enclose two layers of double-sided adhesive material and said biological sample, where present, is housed between said two layers of double-sided adhesive material.

4. A fluidic device for static and/or dynamic biological cultures, comprising:
at least one cartridge according to claim 1;
a support, wherein said support comprises an upper pocket and, optionally, a lower pocket, said two pockets being enclosed between at least two rigid elements, at least one upper rigid element and at least one lower rigid element;
said at least one cartridge being sandwiched between said upper pocket and said lower pocket, if this is present, or between said upper pocket and the at least one lower rigid element if said lower pocket is not present;
wherein said at least one cartridge, said upper, lower pockets, and said rigid elements are crossed by at least one inner hole;
wherein said rigid elements have weakening notches identifying an inner portion and an outer crown;
wherein the area occupied by said cartridge is included in the area identified as the inner portion.

5. The fluidic device according to claim 4, wherein said upper pocket comprises a layer of highly hydrophobic, inert and biocompatible material, defined as a hydrophobic support layer and, optionally, a layer of double-sided adhesive material, defined as a double-sided adhesive support layer, and said lower pocket, where present, comprises a hydrophobic support layer and, optionally, a double-sided adhesive support layer, said hydrophobic support layer facing said hydrophobic support layer of said upper pocket;
wherein, in the absence of one or more of said layers of double-sided adhesive material, said layers are connected together by chemical and/or physical bonding.

6. The fluidic device according to claim 4, wherein said support further comprises a double-sided adhesive frame having a seat adapted to accommodate said at least one cartridge.

7. The fluidic device according to claim 4, which is a static culture device comprising:
a cartridge;
a support comprising:
two rigid elements, wherein said rigid elements each have an inner face, towards the inside of the device, and an outer face, towards the outside of the device;
an upper pocket comprising a layer of highly hydrophobic, inert, and biocompatible material, defined as a hydrophobic support layer, and a layer of double-sided adhesive material, defined as a double-sided adhesive support layer;
a lower pocket comprising a hydrophobic support layer, and a double-sided adhesive support layer, said hydrophobic support layer facing said hydrophobic support layer of said upper pocket;
a double-sided adhesive frame having a seat which accommodates said cartridge.

8. The fluidic device according to claim 7, wherein said support further comprises a well having a distal end and a proximal end, open at the proximal end and optionally open at the distal end, wherein said well is sealingly positioned on the outer face of at least one of said two upper and/or lower rigid elements, wherein said well is positioned on said support so that the opening of said well corresponds with said at least one inner hole.

9. The fluidic device according to claim 4, which is a fluidic device for dynamic culture, comprising:
a cartridge;
a support comprising, sandwiched from the outside towards the inside:
two coverslips which close the access from the external environment to said at least one biological sample when housed in said cartridge;
two layers of double-sided adhesive material;
two rigid elements;
an upper pocket comprising:
a double-sided adhesive support layer;
a hydrophobic support layer;
a lower pocket comprising:
a double-sided adhesive support layer;
a hydrophobic support layer;
a double-sided adhesive frame having a seat which accommodates said cartridge.

10. The fluidic device according to claim 9, further comprising inlet-outlet accesses which put said biological sample in communication with the external environment when housed in said cartridge.

11. The fluidic device according to claim 10, wherein said accesses are four in number, two lateral inlet and/or outlet accesses and two contralateral inlet and/or outlet accesses, wherein said inlet-outlet accesses are coupled to one another and connected by a first and/or a second channel, wherein said first and/or second channels are obtained in the thickness of said double-sided adhesive layers, wherein said first and second channels are in fluid communication with each other only through said biological sample when housed in said cartridge.

12. The fluidic device according to claim 4, having a hydraulic connection which makes use of connectors, wherein said connectors are sealingly positioned outside said fluidic device, at said inlet and/or outlet accesses.

13. A fluidic devices-support station complex comprising:
at least one fluidic device according to claim 4;
a support station;
wherein said support station comprises:
two rigid elements, wherein said rigid elements, which are optionally coverslips, each have an inner face, towards the inside of the support station, and an outer face, towards the outside of the support station;
a frame, interposed between said two rigid elements, having a plurality of seats each of a shape and size adapted to accommodate one of said fluidic devices;
and, at each of said plurality of seats,
an upper pocket housed between said seat and said rigid element,
comprising a hydrophobic support layer and, optionally, a double-sided adhesive support layer;
optionally, a lower pocket housed between said seat and said rigid element comprising a hydrophobic support layer and, optionally, a double-sided adhesive support layer,
wherein said hydrophobic support layers face said seat.

14. A bicompartmental static culture process, comprising:
housing at least one biological sample in the fluidic device according to claim 8;
placing said fluidic device inside a vessel for cell cultures; and
defining an upper chamber, by said well, and a lower chamber,
wherein said lower and upper chambers are in fluid communication with each other only through said at least one biological sample.

15. A bicompartmental dynamic culture process, comprising:
housing at least one biological sample is housed in the fluidic device according to claim 9; and
defining two chambers in said fluidic device, an upper chamber and a lower chamber.

16. A process for employing the fluidic device according to claim 4, comprising:
controlled breaking of said support along said weakening notches; and
subsequently recovering the at least one cartridge and the at least one biological sample housed in the at least one cartridge.
